(19) United States
(12) Patent Application Publication
(10) Pub. No.: US 2007/0276472 A1
(43) Pub. Date: Nov. 29, 2007

(54) Vascular Stent

(76) Inventor: Gianluca Gazza, Princesse Grace (MC)

Correspondence Address: HOGAN & HARTSON LLP;IP GROUP, COLUMBIA SQUARE, 555 THIRTEENTH STREET, N.W., WASHINGTON, DC, 20004, US

(21) Appl. No.: 10/552,030

(22) PCT Filed: May 22, 2003
(86) PCT No.: PCT/IB03/01958

§ 371 Date: Sep. 12, 2006

(30) Foreign Application Priority Data
Apr. 4, 2003 (IB) PCT/IB03/01232

(51) Int. Cl. A61F 2/82; A61L 31/08

(52) U.S. Cl. 623/1.42; 427/2.25; 623/1.46

(57) ABSTRACT

This invention relates to a vascular stent. More particularly, this invention relates to a stent comprising a coating based on a hyaluronic acid polymer in which the said hyaluronic acid polymer is an ester derivative of the hyaluronic acid.

This invention relates to a vascular stent. More particularly, this invention relates to a vascular stent with a polymer coating used in angioplasty to prevent the phenomenon of restenosis.

The fact that stents are widely accepted and used in the cure of coronary occlusions in today's angioplasty is well known. Stents are reticular metal prostheses positioned in the portion of the vessel subject to stenosis, which remain at the site of the lesion after the release system and balloon system have been retracted. Thus the stent compresses the plaque and provides a mechanical support for the vessel wall to maintain the vessel diameter re-established by expansion of the balloon and to prevent collapse of the vessel.

However, the long-term efficacy of the use of intercoronary stents still presents the major problem of post-angioplasty coronary restenosis, that is the phenomenon of reocclusion of the coronary vessel. In fact this phenomenon of restenosis occurs in 15-30% of patients subjected to stent angioplasty, as described for example in Williams D O, Holubkov R, Yeh W, et al. “Percutaneous coronary interventions in the current era are compared with 1985-1986: The National Heart, Lung and Blood Institute Registries”, Circulation 2000; 102: 2945-2951.

The stenosis caused by insertion of the stent is due to hyperplasia of the newly-formed intima. In particular, mechanical damage caused to the artery wall by the stent and the foreign-body reaction induced by the presence of the stent give rise to a chronic inflammatory process in the vessel. This phenomenon in turn gives rise to the release of cytokines and growth factors which promote activation of the proliferation and migration of smooth muscle cells (SMC). The growth of these cells together with the production of extra-cellular matrix give rise to an increase in the cross-section of the vessel occupied by the neointema and therefore a process of reducing the lumen of the vessel, bringing about the abovementioned restenosis.

Numerous pharmacological approaches attempted via the systemic route have not yielded useful results in terms of reducing the level of restenosis after angioplasty. The problem with this method of administration can in fact be identified in the low concentration of the pharmacologically active ingredient which reaches the stenotic lesion.

An alternative approach to prevent the problem of restenosis, which brings about greater release of active ingredient in the zone requiring treatment, is provide d by the use of coated stents, used as a local source capable of releasing drugs (DES, drug eluting stent). For example, in the article by Takeshi Suzuki et al. “Stent-Based Delivery of Sirolimus Reduces Neointimal Formation in a Porcine Coronary Model” Circulation 2001; 104: 1188-1193, stents coated with a non-degradable polymer matrix based on poly-n-butyl methacrylate and polyethylene-vinyl acetate containing a therapeutic concentration of active ingredient, designed to reduce hyperplasia of the neointima, are described.

Polymer coatings for the release of active ingredients in which the polymers may be of a degradable or non-degradable nature are known. These however only ever have an inert function, that is they are restricted to acting as reservoirs for the active ingredient and therefore controlling its rate of release, without however being able to act themselves in any way on the atherosclerotic lesion.

Contrary to what has just been said, there are however in nature also polymers which are capable of playing an active role in control of the processes in restenosis. The useful properties of hyaluronic acid, a natural polysaccharide which is found in molecular form in the tissues of various species of mammals, are particularly well known in the biomedical field. Hyaluronic acid in fact has appreciable properties in reducing the foreign-body reaction and therefore the consequent process of inflammation. In addition to this hyaluronic acid plays a fundamental part in the processes of restenosis, as a result of its specific interaction with smooth muscle cells (SMC) and endothelial cells. As a result of these features it has been shown in animal models that the exposure of arterial lesions to high concentrations of hyaluronic acid gives rise to a significant reduction in the growth of neointima.

However, it is not immediately possible to apply hyaluronic acid as a coating and reservoir of active ingredient to a stent. In fact hyaluronic acid is extremely soluble in water and is therefore immediately dissolved and moved away from the site of the lesion. Its immediate dissolution therefore gives rise to immediate release of all of any active ingredient which may have been incorporated, with the risk of exposing the harmed site to excessive and toxic doses of the active ingredient, and with an absolute impossibility of controlling the kinetics of release of active ingredient from the natural polymer.

In order to overcome these disadvantages various examples of techniques to immobilise hyaluronic acid on the surface of a stent have been reported. In general, in the methods of surface modification already described in the literature, the hyaluronic acid is covalently bound to the surface of the stent. However, with this approach the natural polymer is no longer available to be released in high concentrations which are therapeutically effective at the site of the implant. In addition to this, because the immobilisation reaction takes place at the interface between the material which has to be coated and the hyaluronic acid, the thickness of the polymer layer is restricted to a single molecular layer, which is certainly not suitable as a reservoir for a therapeutically effective quantity of active ingredient. It therefore follows that the quantity of hyaluronic acid which might be available and the quantity of active ingredient which might be capable of incorporation are extremely small and therefore insufficient to prevent the phenomenon of restenosis.

Hyaluronic acid can however be applied as a coating in more significant thicknesses, of the order of a few microns, through a reaction which cross-links the hyaluronic acid itself. This cross-linking reaction is for example carried out with a polyurethane. This cross-linking process is not however suitable for application in the context of coatings for stents. In fact this has proved to be difficult to implement on a device having a complex geometry such as a vascular stent, it has given rise to collateral effects due to the cross-linking agent, such as for example the collateral effects due to polyurethane, and above all the hyaluronic acid immobilised by cross-linking has lost its biochemical properties and is therefore no longer available to act actively in the control of restenosis.

Finally another known approach to reduce the solubility of hyaluronic acid is that of forming mixtures with natural or synthetic materials with which the stent is then coated. An example is the coating of a stent with the reabsorbable film Seprafilm® from the Genzyme company. This film consists of a mixture of hyaluronic acid and carboxymethylcellulose. However these films also have the major disadvantage of the collateral effects of carboxymethylcellulose on the inflammatory response at the stenotic lesion.

The need for the development of a stent which can be used in angioplasty and which is capable of effectively preventing the phenomenon of restenosis therefore appears to be obvious.

As a consequence, the technical problem underlying this invention is that of providing a new stent which does not have all the disadvantages of the stents in the known art described above.

This problem is resolved by a stent according to this invention which comprises a polymer coating constituting ester derivatives of hyaluronic acid as described in the appended claims.

Other advantages and characteristics of the present invention will become clear from the following detailed description which is given with reference to the appended drawings which are provided purely by way of non-limiting example and in which:

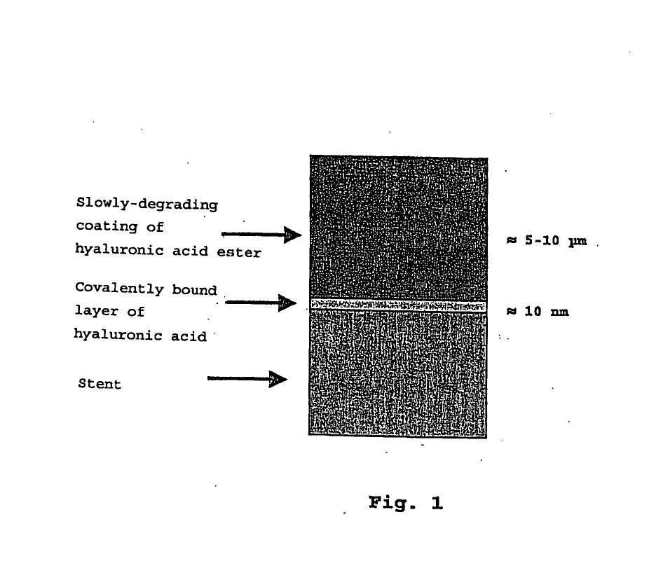

FIG. 1 shows a diagram in cross-section of a detail of the polymer coating for the stent according to an embodiment of this invention.

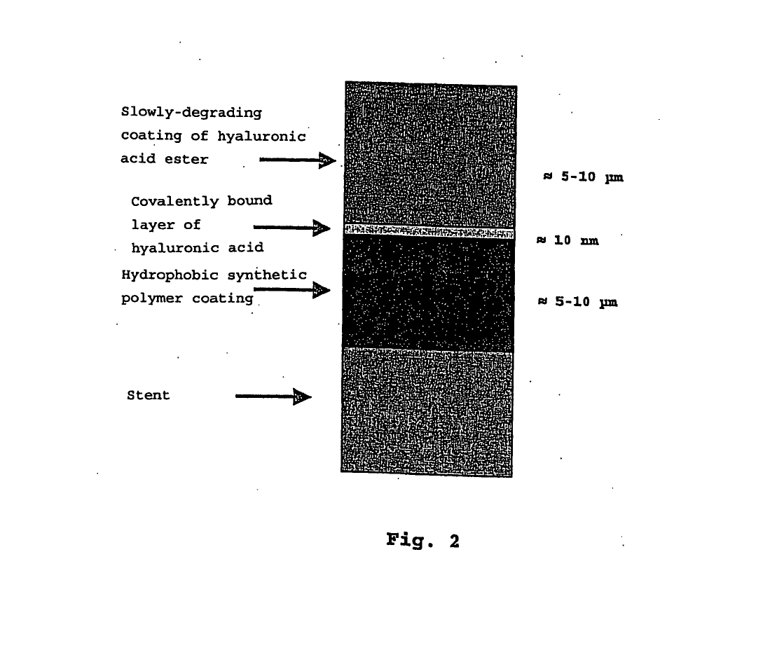

FIG. 2 shows a diagram in cross-section of a detail of the polymer coating for the stent according to another embodiment of this invention.

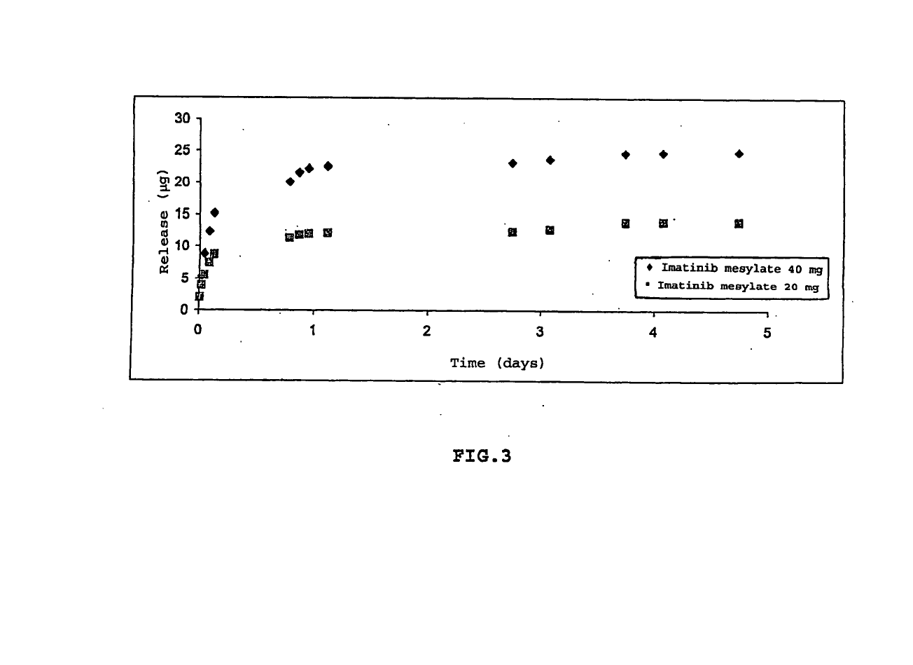

FIG. 3 shows a graph indicating the release curve for the active ingredient from the polymer coating of the stent according to the embodiment illustrated diagrammatically in FIG. 1 and the effect on the release of the concentration of active ingredient in that coating.

Hyaluronic acid esters which are suitable for coating the stent according to this invention are for example those described in European patent EP 216453 by the Fidia Advanced Biopolymers company, included here for reference.

These compounds are hyaluronic acid esters in which all or part of the carboxyl groups are esterified with alcohol groups selected from those in the aliphatic, arylaliphatic, cycloaliphatic and hetrocyclic series.

Alcohols of the aliphatic series used to esterify the carboxyl groups of the hyaluronic acid are selected from straight or branched saturated or unsaturated alcohols having from 2 to 12 carbon atoms, optionally substituted with one or more groups selected from hydroxide, amine, aldehyde, mercaptan or carboxyl groups or groups derived from these such as for example esters, ethers, acetals, ketals, thioethers, thioesters, carbamides.

When the alcohol is a saturated and non-substituted aliphatic alcohol it is preferably selected from methyl, ethyl, propyl, isopropyl, normal butyl, isobutyl, ter-butyl, amyl or pentyl alcohol.

When the alcohol is a bivalent aliphatic alcohol it is preferably selected from the alcohols ethylene glycol, propylene glycol, butylene glycol, or if it is a trivalent aliphatic alcohol it is preferably glycerine.

When the aliphatic alcohol is an amino alcohol, this is preferably selected from aminoethanol, aminopropanol, aminobutanol or their dimethylene- or diethyleneamine derivatives, piperidine ethanol, pyrrolidine ethanol, piperazine ethanol.

When the alcohol is a carboxy alcohol, it is preferably selected from lactic, tartaric, maleic and glycolic acids.

Finally, when the alcohol is an unsaturated aliphatic alcohol it is preferably an allyl alcohol.

Alcohols of the arylaliphatic series used to esterify the carboxyl groups of the hyaluronic acid are preferably selected from those having a benzene optionally substituted by from 1 to 3 methyl or hydroxyl groups or halogen atoms, in particular fluorine, chlorine, bromine and iodine, and in which the aliphatic chain has from 1 to 4 carbon atoms and is optionally substituted by one or more groups selected from primary amine groups, mono- or dimethylates or pyrrolidine or piperidine groups.

Preferably the alcohols of the arylaliphatic series used to esterify the carboxyl groups of the hyaluronic acid are benzyl alcohol and phenylethyl alcohol.

Alcohols of the cycloaliphatic series used to esterify the carboxyl groups of the hyaluronic acid are preferably selected from those mono- or polycyclic alcohols containing from 3 to 34 carbon atoms and optionally containing from 1 to 3 heteroatoms selected from O, S, N and optionally substituted with one or more groups selected from those listed for the aliphatic alcohols.

In particular, of the monocyclic cycloaliphatic alcohols, those of particular interest for this invention are those containing from 5 to 7 carbon atoms, optionally substituted with one or more groups selected from hydroxyl, methyl, ethyl, propyl or isopropyl. For example, the alcohols cyclohexanol, cyclohexandiol, inositol and menthol are used.

The degree of esterification of the ester derivatives of hyaluronic acid with the abovementioned alcohols can vary depending upon the characteristics which it is desired to impart to the coating on the stent, for example a coating having a greater or a lesser lipophilic or hydrophilic character.

In general in fact a higher degree of esterification increases the lipophilic nature of the ester derivative and therefore reduces its solubility in water. This makes it possible to obtain stents according to this invention with a coating which degrades slowly at the site of the stenosis, therefore having an action which is prolonged over time, in comparison with a coating of hyaluronic acid which is instead immediately dissolved and carried away from the site of the lesion.

For the purposes of this invention the degree of esterification of the ester derivatives of hyaluronic acid varies from 50% to 100% of the carboxyl groups of the hyaluronic acid being esterified with alcohol groups of the abovementioned alcohols. Preferably the degree of esterification varies from 70% to 100% of the carboxyl groups of the hyaluronic acid.

In the preferred embodiment of this invention the stent is coated with a product obtained by the esterification of hyaluronic acid with benzyl alcohol.

Even more advantageously the derivative obtained from total esterification of the hyaluronic acid with benzyl alcohol, or the derivative obtained by esterification of 75% of the residual carboxyls of the hyaluronic acid with benzyl alcohol are used.

These products have proved to be particularly useful for the production of coatings for stents according to this invention. In fact the process of the esterification of hyaluronic acid advantageously makes it possible to obtain a polymer derivative which is capable of controlling the solubility and release of the hyaluronic acid itself in water. In fact the process of attack on the ester by water molecules comprises de-esterification of the ester derivative with the consequent release of hyaluronic acid and the alcohol group.

In the particular embodiment in which this alcohol group is benzyl alcohol, the hyaluronic acid ester is also biocompatible and has no collateral effects.

Degradation of the ester derivative therefore brings about progressive release of hyaluronic acid, which is therefore dissolved and made available to act actively at the site of the lesion.

In particular, the abovementioned preferred products, that is the derivative from the total esterification of hyaluronic acid with benzyl alcohol or that obtained by esterification of 75% of the carboxyl groups of the hyaluronic acid with benzyl alcohol, degrade in water in a time of longer than one month and in a time of within two weeks respectively.

It was also found, surprisingly, that these ester derivatives of hyaluronic acid form a homogeneous coating film on the metal stent which adheres well to the reticular surface of the stent.

The stent obtained according to this invention therefore comprises a coating which is also capable of effectively associating itself with a pharmacologically active ingredient.

According to this invention the active ingredients selected for association with the polymer coating are active ingredients having an anti-inflammatory, anti-proliferative and anti-migratory action, and immunosuppressants.

Even more preferably the active ingredient associated with the polymer coating of the stent according to this invention is imatinib mesylate, that is 4-[(4-methyl-1-piperazinyl)methyl]-N-[4-methyl-3-[[4-(3-pyridinyl)-2-pyrimidinyl]amino]-phenyl]benzamide methane sulphonate, marketed under the name Glivec® by Novartis company.

The quantity of active ingredient which has to be associated with the hyaluronic acid ester coating varies according to the class of active ingredient.

When the active ingredient is an active ingredient having an anti-inflammatory action it is preferably associated with the polymer coating in a quantity of between 0.001 mg and 10 mg.

When the active ingredient is an active ingredient having an anti-proliferative action it is preferably associated with the polymer coating in a quantity of between 0.0001 mg and 10 mg.

When the active ingredient is an active ingredient having an antimigratory action it is preferably associated with the polymer coating in a quantity of between 0.0001 mg and 10 mg.

When the active ingredient is an immunosuppressant it is preferably associated with the polymer coating in a quantity of between 0.0001 mg and 10 mg.

More particularly, when the active ingredient is imatinib mesylate (Glivec®) it is associated with the polymer coating in a quantity of between 0.001 mg and 10 mg.

The hyaluronic acid esters used to coat the stent according to this invention also have some solubility in organic solvents, unlike hyaluronic acid, in particular in dipolar aprotic organic solvents.

In particular the esters of hyaluronic acid have good solubility in dimethyl sulphoxide, N-methylpyrrolidone and dimethyl formamide. These solvents can also dissolve different active ingredients.

Some esters are also soluble in the low-boiling-point solvent 1,1,1,3,3,3-hexafluoro-2-propanol (hexafluoroisopropanol), which in turn is a solvent for imatinib mesylate. The boiling point of hexafluoroisopropanol is 59° C. at ambient pressure, a feature which makes it possible to remove the solvent at temperatures compatible with stability of the active ingredient.

These solubility properties are another advantage of this invention. In fact they make it possible to apply the hyaluronic acid derivative and the active ingredient directly from a single common solution onto the surface of the stent at the desired concentrations through the dip coating technique. Removal of the solvent by evaporation, if necessary under vacuum, makes it possible to obtain a thin film, of a thickness which can be controlled through the main process parameters, adhering to the surface of the stent.

The thickness of the hyaluronic acid ester coating on the stent varies from 0.5 microns to 40 microns, preferably between 1 and 30 microns, and even more preferably between 5 and 10 microns.

Unlike a similar stent comprising a film of hyaluronic acid, which is immediately dissolved in an aqueous environment with consequent complete and immediate release of the active ingredient and the hyaluronic acid, the stent according to this invention comprises a film which undergoes a process of degradation in an aqueous environment, and therefore a release of active ingredient and hyaluronic acid molecules governed by the properties of the ester. In fact the period for degradation and consequent release of the hyaluronic acid and the active ingredient can be controlled through the film thickness and the intrinsic properties of the polymer, in particular through the degree of esterification.

There is therefore a release of active ingredient and a release of hyaluronic acid over a prolonged time in the vicinity of the stenotic lesion, that is a period of time equal to the degradation time of the polymer coating based on the hyaluronic acid ester derivative. In particular, in the preferred embodiments mentioned above, obtained by the esterification of 100% and 75% of the carboxyl groups with benzyl alcohol respectively, the active ingredient is released for a period of more than 1 month or for a period of up to 2 weeks respectively.

It therefore appears obvious that with this invention it is possible to obtain advantageously stents comprising a polymer coating which is capable of preserving all the intrinsic biological and therapeutic properties of the hyaluronic acid itself, which has low solubility in an aqueous environment so that it is not immediately removed from the surface of the stent, and which has a thickness compatible with an association with an active ingredient which will be delivered and released in a controlled way and over periods which are clinically useful.

The stent according to this invention therefore has the further advantage that it can combine the effect of the active ingredient at cellular level at the site of the lesion with that of reducing the inflammation process and controlling cellular migration of the hyaluronic acid itself, over a prolonged and controllable time, so as to be able to effectively prevent the phenomenon of restenosis.

In a particularly preferred embodiment of this invention, illustrated diagrammatically in FIG. 1, the layer of hyaluronic acid ester associated with the active ingredient is applied to a stent which has been first coated with a thin layer of hyaluronic acid bound to the surface of the stent covalently. The process of immobilising the hyaluronic acid layer on the surface of the stent through covalent bonds can be carried out in accordance with the method described in U.S. Pat. No. 6,129,956 in the name of Fidia Advanced Biopolymers and as shown below in Example 9.

The thickness of the layer of hyaluronic acid covalently bound to the surface of the stent varies from 1 nm to 20 nm, preferably 10 nm.

In this way, when the coating of hyaluronic acid ester derivative is degraded in the vicinity of the stenotic lesion, releasing hyaluronic acid and the active ingredient, the tissue of the vessel remains advantageously in contact with a layer of hyaluronic acid, which is more biocompatible and biotolerable than the steel surface of the stent itself.

Another embodiment of this invention provides a stent which has a second coating of a synthetic polymer of a hydrophobic nature in addition to the coating of the hyaluronic acid ester derivative described above.

Preferably the said synthetic polymer coating of a hydrophobic nature is applied directly to the surface of the stent and then in turn coated by the coating of hyaluronic acid ester derivative previously described in this invention.

The level of the hydrophobic nature of the polymers constituting this second coating is measured using the technique of the contact angle with water. In particular the synthetic polymers of a hydrophobic nature which are suitable for use in forming the second polymer coating on the stent have a contact angle with water of more than 60° C.

These polymers having a hydrophobic nature are preferably selected from polymethylmethacrylate, polybutylmethacrylate, polyisobutylmethacrylate, olefinic polymers, polybutadiene, polyisoprene, poly(acrylonitro-butadiene-styrene) or Polyvinyl acetate.

In an even more preferred embodiment the synthetic polymer having a hydrophobic nature is polystyrene.

Furthermore, the second synthetic polymer coating is in turn capable of being effectively associated with a pharmacologically active ingredient. In this way therefore it carries out the role of an inert coating, underlying the first active coating of hyaluronic acid derivatives, capable of acting as a second reservoir of active ingredient and therefore of also subsequently controlling the rate of release of the said active ingredient associated with it at the site of the lesion.

The classes of active ingredients preferably associated with the said polymer coating of a hydrophobic nature, and the quantities of active ingredient associated therewith, are the same as described previously for the coating obtained from hyaluronic acid ester derivatives.

Identical or different active ingredients, depending upon the therapeutic objective sought, can therefore be associated with the two polymer coatings, that of a hydrophobic nature and that based on the hyaluronic acid ester derivative, on the same stent. Also the corresponding quantities of active ingredient associated with the two coatings on the stent may be the same or different according to therapeutic needs.

Application of the polymer coating having a hydrophobic nature and the active ingredient associated with it can be applied to the stent in a manner similar to that first described for application of the coating of hyaluronic acid derivative. The hydrophobic polymer and the active ingredient are dissolved or suspended in the same organic solvent to form a single common solution or suspension. Solvents suitable for this purpose should have low boiling points, with a boiling point at ambient pressure of below 100° C. and preferably below 80° C. Preferably the said organic solvents are selected from dichloromethane, methylene chloride, acetone, aliphatic hydrocarbons or cyclohexane, preferably dichloromethane.

Through evaporation of the said solvent a coating of variable thickness, depending upon the process parameters, adhering to the surface of the stent is this obtained. The coating based on the ester derivative of hyaluronic acid is then subsequently applied to the stent pre-treated in this way.

The thickness of the hydrophobic synthetic polymer coating on the stent varies from 0.5 microns to 40 microns, preferably between 1 and 30 microns, and even more preferably between 5 and 10 microns.

It therefore appears obvious that the further advantage of this embodiment of the stent is that of being able to modulate the rate of release of the active ingredient through the double coating on the stent, further extending release of the said active ingredient over time and therefore extending its pharmacological action on the stenotic lesion. In fact, with this embodiment, at the atherosclerotic lesion there is a first double action due to coupling of the effect of the active ingredient and the hyaluronic acid, both released by the process of degradation of the hyaluronic acid ester derivative coating, and subsequently the effect of the active ingredient released by the second inert polymer coating.

In this way the therapeutic effect can be prolonged at the site of the lesion for a time equal to the release time for the active ingredient from the polymer coating of a hydrophobic nature.

In the particular embodiment in which the polymer coating having a hydrophobic nature is based on polystyrene the release period for the active ingredient is further extended by a period of one month.

Similarly to what has been described above and as illustrated diagrammatically in FIG. 2, a particularly preferred embodiment of this two-layer coating for the stent provides that the underlying polymer layer of a hydrophobic nature is coated with a thin layer of hyaluronic acid which is chemically bound in a covalent manner. The coating of hyaluronic acid ester derivative is then applied to this layer of covalently bound hyaluronic acid. In this way, when the upper layer of hyaluronic acid ester has degraded, the tissue of the vessel is not exposed to the synthetic polymer, but to a layer of hyaluronic acid.

The process of forming a covalent bond between the polymer coating of a hydrophobic nature and the layer of hyaluronic acid is carried out for example in accordance with the method described in the aforementioned U.S. Pat. No. 6,129,956 in the name of Fidia Advanced Biopolymers.

The thickness of the layer of hyaluronic acid covalently bound to the surface of the polymer coating of hydrophobic nature varies from 1 nm to 20 nm, preferably 10 nm.

The invention is further described through the following illustrative and non-restrictive examples of the same, from which the features and advantages of this invention will be even more obvious.

### EXAMPLE 1

### Formation of a Film of Hyaluronic Acid Ester Derivative of Different Thicknesses Obtained by Total Esterification of the Carboxyl Groups with Benzyl Alcohol.

A Laserskin membrane, manufactured by the company Fidia Advanced Biopolymers, constructed in particular using HYAFF 11®, was used to form a film of hyaluronic acid ester derivative obtained from total esterification of the carboxyl groups with benzyl alcohol (a product having the trade name of HYAFF 11®). Some fragments having a total weight of 70 mg were cut off from the membrane. These were dissolved in 3 ml of dimethylsulphoxide (DMSO). Dissolution took place at ambient temperature over 1 hour. When a homogeneous solution was formed three aliquots of solution, 0.5 ml, 1 ml and 1.5 ml, were taken respectively. DMSO was added to each aliquot of solution in a quantity such as to make up each solution to 3 ml and three solutions A, B and C respectively were obtained in this way. The three solutions so obtained were poured into polystyrene Petri dishes and placed within a stove at 60° C. where they remained until the solvent had completely evaporated. The film deposited on the base of the Petri dish was recovered and its thickness was evaluated by observing through a scanning electron microscope. Observation yielded the following results shown in Table 1, which are expressed as the mean value of four measurements.

TABLE 1

| Solution | Thickness (μm) |
|---|---|
| A | 11 ± 6 |
| B | 23 ± 10 |
| C | 38 ± 8 |

### EXAMPLE 2

### Application of the Film According to Example 1 to a Stainless Steel Stent

Solution A obtained according to example 1 was used. A stainless steel stent of dimensions 13 mm was immersed into and removed from the solution contained in a beaker and transferred to a stove at 60° C. under vacuum. After drying the stent was immersed in a solution of toluidene blue, which is a stain capable of colouring hyaluronic acid, in order to evaluate film formation. The existence and uniformity of the colour was then observed. The test thus confirmed the presence of a film of HYAFF 11® on the surface of the stent, and its uniform distribution.

### EXAMPLE 3

### Incorporation of an Active Ingredient in the HYAFF 11® Film and its Release

Solutions of HYAFF 11® in DMSO were prepared as described in Example 1. 10 mg of the active ingredient imatinib mesylate, obtained from the drug Glivec® following dissolution in water, filtration to remove insoluble excipients, and evaporation of the water, were added to the solution. After dissolution the solution was placed in a stove and the solvent was evaporated.

Cytotoxicity tests were carried out using Balb/3T3 cells to evaluate the presence of the active ingredient. 0.5 cm² portions of film were placed in a Petri dish containing a confluent layer of such cells. For each of the concentrations of the various samples of the said hyaluronic acid ester derivative A, B and C in Example 1, a control comprising the said hyaluronic acid ester derivative A, B and C without the active ingredient was prepared. The effect on the cells was evaluated after one day's contact and expressed using a cytotoxicity scale with values from 0 to 5; value 0 indicates the absence of any cytotoxic effect, while value 5 indicates death of all the cells. Table 2 below shows the results so obtained.

TABLE 2

| Sample | Cytotoxic effect |
|---|---|
| A | 5 |
| A Control | 0 |
| B | 3 |
| B Control | 0 |
| C | 3 |
| C Control | 0 |

From the results obtained it is clear that the cytotoxic effect previously established for the pure active ingredient confirms that the active ingredient is released from the HYAFF 11® film on the stent.

### EXAMPLE 4

### Monitoring of the Concentration of Active Ingredient Associated with the HYAFF 11® Film

HYAFF 11® films of type A were obtained as in example 3 above, but different quantities of active ingredient, 10 mg, 5 mg, 1 mg and 0.1 mg, were incorporated. Cell culture tests were performed as reported in Example 3 and the results shown in Table 3 were obtained.

TABLE 3

| Quantity of active | |
|---|---|
| ingredient in the type A | |
| HYAFF 11 ® film | Cytotoxic effect |
| 10 mg | 5 |
| 5 mg | 5 |
| 1 mg | 1 |
| 0.1 mg | 0 |

This experiment demonstrates that it is possible to control the concentration of active ingredient in the film, thus controlling the time period of the effect on the cells, from a toxic effect to a sub-toxic effect.

### EXAMPLE 5

### Incorporation of Active Ingredient into the HYAFF 11® Film and its Release Over Time

A HYAFF 11® film of type A as described in Example 3 and a control film without active ingredient were prepared. The films were then subdivided into 0.5 cm² portions. Four portions of each film were immersed in physiological solution for periods of one day, two days, one week and two weeks respectively. At the end of the immersion period the samples were removed from the physiological solution and subjected to the cytotoxicity test under the same conditions as reported in Example 3. The results obtained are shown below in Table 4.

TABLE 4

| Residence time | Cytotoxic effect |
|---|---|
| 1 day | 5 |
| 2 days | 5 |
| 1 week | 4 |
| 2 weeks | 3 |

The controls without active ingredient did not however show any signs of cytotoxicity.

These data demonstrate that the active ingredient incorporated in the HYAFF 11® film is released slowly even after it has remained in an aqueous environment for 2 weeks, confirming the active ingredient reservoir function of the layer of hyaluronic acid ester derivative.

### EXAMPLE 6

### Manufacture of a Stent with a Coating of HYAFF 11® and Release of the Active Ingredient Associated with that Coating

A number of stents were prepared as described in Example 2, in particular 10 mg of the active ingredient imatinib mesylate were added to solution A of HYAFF 11® prepared in accordance with Example 1. The stents were then immersed in physiological solution for 0, 1 and 2 days and 1 week respectively. The experiment described in Example 5 was repeated with the stents prepared in this way. The results shown in Table 5 below were obtained.

TABLE 5

| Residence time | Cytotoxic effect |
|---|---|
| 1 day | 5 |
| 2 days | 5 |
| 1 week | 4 |

This experiment again confirms that active ingredient is released over time from the stent coated with HYAFF 11® film.

### EXAMPLE 7

### Manufacture of a Stent with HYAFF 11® Coating Using a Low-Boiling-Point Solvent and Release of the Active Ingredient Associated with that Coating

Some stents with HYAFF 11® were prepared as described in general in example 2, but using hexafluoroisopropanol as solvent.

A solution of HYAFF 11® in hexafluoroisopropanol to which the active ingredient imatinib mesylate was added was therefore prepared for this purpose. In particular a solution containing 5 cc of hexafluoroisopropanol, 40 mg of HYAFF 11® and 20 mg of imatinib mesylate was prepared. Removal of the solvent after the stents had been immersed in the solution took place in a vacuum stove at 25° C.

The stents were then immersed in physiological solution for 0, 1 and 2 days and for 1 week respectively. The experiment described in example 5 was repeated with the stents prepared in this way. The following results shown in Table 6 below were obtained.

TABLE 6

| Residence time | Cytotoxic effect |
|---|---|
| 1 day | 5 |
| 2 days | 5 |
| 1 week | 4 |

This experiment again confirms that the active ingredient is released over time from the stent coated with HYAFF 11® film.

### EXAMPLE 8

### Manufacture of a Stent with a HYAFF 11® Coating and a Second Coating of Synthetic Polymer of a Hydrophobic Nature and Release of the Active Ingredient Associated with that Coating.

A number of stents were prepared as described in general in Example 7, but acting on the pre-treated stents as follows:

A suspension of imatinib mesylate in a 2% solution of polystyrene in dichloromethane was prepared. The stent was coated by immersion in the solution and the solvent was subsequently removed in a vacuum stove at 30° C. The process was repeated 3 times.

For comparison purposes, a number of stents were prepared in which the same steps were carried out using a solution of HYAFF 11® and imatinib mesylate.

The stents were then immersed in physiological solution for 0, 1 and 2 days and for 1 week and 3 weeks respectively. The experiment described in Example 5 was repeated with the stents prepared in this way. The results shown in Table 7 below were obtained.

TABLE 7

| | | Cytotoxic effect |
|---|---|---|
| | | (HYAFF 11 ®/imatinib |
| | | mesylate and |
| | Cytotoxic effect | hydrophobic |
| Residence | (HYAFF 11 ®/imatinib | polymer/imatinib |
| time | mesylate) | mesylate) |
| 1 day | 5 | 5 |
| 2 days | 5 | 5 |
| 1 week | 4 | 4 |
| 3 weeks | 1 | 3 |

This experiment again confirms that active ingredient is released from the coated stent over time and is evidence that the presence of a hydrophobic polymer containing active ingredient can assist and extend the release of active ingredient at the site of the lesion.

### EXAMPLE 9

### Manufacture of a HYAFF 11® Coating on a Stent Pre-Coated with a Layer of Covalently Bound Hyaluronic Acid and Release of Active Ingredient Associated with this Coating.

A number of steel stents were coated with a layer of hyaluronic acid, covalently bound to the surface of the stent, in accordance with the method described in U.S. Pat. No. 6,129,956 (in the name of Fidia Advanced Biopolymers). More particularly, the stents were subjected to plasma treatment with air plasma for 1 minute in a Europlasma reactor. The stents were then immersed in a 0.5% aqueous solution of polyethyleneimine (PEI, Sigma) for 2 hours at ambient temperature. The stents were then repeatedly washed and immersed in a solution of 0.5% hyaluronic acid (SIGMA) containing 1% of N-hydroxysuccinimide (SIGMA) and 1% of dimethylamino propylethylcarbodiimide (EDC, Sigma). The bonding reaction continued through the night, at ambient temperature. On the next day the stents were carefully washed.

The stents pre-treated in this way were subjected to coating by immersion in a solution of HYAFF 11® in hexafluoroisopropanol as generally described in Example 7. In particular two solutions, a first comprising 5 ml of hexafluoroisopropanol, 40 mg of HYAFF 11® and 20 mg of imatinib mesylate, and a second identical solution but containing twice the concentration of imatinib mesylate, that is 40 mg, were used.

Each stent so obtained was placed in a test tube containing 1 mL of physiological solution at 37° C. in order to carry out the investigations on the release of imatinib mesylate from the HYAFF 11® coating. The solution was removed and examined using a Unicam UV-Visible spectrophotometer at specific times. The concentration of imatinib mesylate released by the stent was calculated by measuring the absorbance of the solution at a wavelength of 251 nm. The correlation between absorbance and imatinib mesylate concentration was established by plotting a calibration curve, that is by measuring the absorbance of solutions having a known concentration of imatinib mesylate in normal saline.

The experiments on the stents obtained in accordance with this experiment from a solution containing 20 mg of imatinib mesylate or the solution containing 40 mg of imatinib mesylate respectively provided the two release curves illustrated in FIG. 3.

1. A stent comprising a coating based on a polymer of hyaluronic acid characterized in that the said hyaluronic acid polymer is an ester derivative of hyaluronic acid.

2. A stent according to claim 1 in which the said hyaluronic acid ester derivative has all or some of the carboxyl groups of the hyaluronic acid esterified with alcohols selected from those of the aliphatic, arylaliphatic, cycloaliphatic and heterocyclic series.

3. A stent according to claim 2, in which:
when the said alcohols are of the aliphatic series they are selected from straight or branched saturated or unsaturated alcohols having from 2 to 12 carbon atoms, optionally substituted with one or more groups selected from hydroxide, amine, aldehyde, mercaptan or carboxyl groups or groups derived from these such as for example esters, ethers, acetals, ketals, thioethers, thioesters, carbamides; in particular when the said alcohols are saturated aliphatic alcohols they are selected from methyl, ethyl, propyl, isopropyl, normal butyl, isobutyl, ter-butyl, amyl or pentyl alcohols; when the said alcohols are bivalent aliphatic alcohols they are selected from the alcohols ethylene glycol, propylene glycol, butylene glycol, and when the alcohol is a trivalent aliphatic alcohol it is preferably glycerine; when the said alcohols are amino alcohols, they are selected from aminoethanol, aminopropanol, aminobutanol and their dimethylene- or diethyleneamine derivatives, piperidine ethanol, pyrrolidine ethanol or piperazine ethanol; when the said alcohols are carboxy alcohols, they are selected from lactic, tartaric, maleic or glycolic acids; when the said alcohols are unsaturated aliphatic alcohols they are preferably allyl alcohols,
when the said alcohols are of the arylaliphatic series they are selected from those having a benzene optionally substituted with from 1 to 3 methyls or hydroxyls or halogen atoms, in particular fluorine, chlorine, bromine and iodine, and in which the aliphatic chain has from 1 to 4 carbon atoms and is optionally substituted by one or more groups selected from primary amine groups, mono- or dimethylated groups or from pyrrolidine or piperidine groups, in particular they are benzyl alcohol or phenylethyl alcohol,
when the said alcohols are of the cycloaliphatic series they are selected from those mono- or polycyclic alcohols containing from 3 to 34 carbon atoms and optionally containing from 1 to 3 hetero atoms selected from O, S, N and optionally substituted with one or more groups selected from hydroxyl, amine, aldehyde, mercaptan or carboxyl groups or groups derived from these such as for example esters, ethers, acetals, ketals, thioethers, thioesters, carbamides; in particular when the said cycloaliphatic alcohols are monocyclic they are selected from those containing from 5 to 7 carbon atoms, optionally substituted with one or more groups selected from hydroxyl, methyl, ethyl, propyl, isopropyl, and in particular they are cyclohexanol, cyclohexandiol, inositol or menthol.

4. A stent according to claim 1, in which the degree of esterification of the said hyaluronic acid ester derivative varies from 50% to 100% of the carboxyl groups in the hyaluronic acid.

5. A stent according to claim 4 in which the degree of esterification varies from 70% to 100% of the carboxyl groups in the hyaluronic acid.

6. A stent according to claim 1, in which the alcohol is benzyl alcohol and the degree of esterification is equal to 100% of the carboxyl groups in the hyaluronic acid.

7. A stent according to claim 1, in which the alcohol is benzyl alcohol and the degree of esterification is equal to 75% of the carboxyl groups in the hyaluronic acid.

8. A stent according to claim 1, in which a pharmacologically active ingredient is associated with the said hyaluronic acid polymer coating.

9. A stent according to claim 8 in which the said active ingredient associated with the said hyaluronic acid polymer coating is selected from active ingredients having an anti-inflammatory, antiproliferative or antimigratory action and/or immunosuppressants.

10. A stent according to claim 8 in which the said active ingredient is 4-[(4-methyl-1-piperazinyl)methyl]-N-[4-methyl-3-[[4-(3-pyridinyl)-2-pyrimidinyl]amino]-phenyl]benzamide methane sulphonate.

11. A stent according to claim 9, in which when the active ingredient is an active ingredient having an anti-inflammatory action it is associated with the hyaluronic acid polymer coating in a quantity of between 0.001 mg and 10 mg.

12. A stent according to claim 9, in which when the active ingredient is an active ingredient having an anti-proliferative action it is associated with the hyaluronic acid polymer coating in a quantity of between 0.0001 mg and 10 mg.

13. A stent according to claim 9, in which when the active ingredient is an active ingredient having an anti-migratory action it is associated with the hyaluronic acid polymer coating in a quantity of between 0.0001 mg and 10 mg.

14. A stent according to claim 9, in which when the active ingredient is an immunosuppressant it is associated with the hyaluronic acid polymer coating in a quantity of between 0.0001 mg and 10 mg.

15. A stent according to claim 10, in which when the active ingredient is 4-[(4-methyl-1-piperazinyl)methyl]-N-[4-methyl-3-[[4-(3-pyridinyl)-2-pyrimidinyl]amino]-phenyl]benzamide methane sulphonate, this is associated with the hyaluronic acid polymer coating in a quantity of between 0.001 mg and 10 mg.

16. A stent according to claim 1, in which the thickness of the hyaluronic acid polymer coating on the stent varies from 0.5 microns to 40 microns, preferably between 1 and 30 microns, even more preferably between 5 and 10 microns.

17. A stent according to claim 1, in which the active ingredient and the hyaluronic acid are released from the hyaluronic acid polymer coating over a prolonged time.

18. A stent according to claim 6, in which the active ingredient and the hyaluronic acid are released from the hyaluronic acid polymer coating in a time exceeding one month.

19. A stent according to claim 7, in which the active ingredient and the hyaluronic acid are released from the hyaluronic acid polymer coating within two weeks.

20. A stent comprising a layer of hyaluronic acid covalently bound to the surface of the stent itself and a coating of hyaluronic acid polymer as described in claim 1.

21. A stent according to claim 1, further comprising a second coating of a polymer having a hydrophobic nature with which a pharmacologically active ingredient is associated.

22. A stent according to claim 21 in which the said polymer coating having a hydrophobic nature is applied directly to the surface of the stent, beneath the said coating based on hyaluronic acid ester polymer.

23. A stent according to claim 21, in which the said polymer having a hydrophobic nature has a contact angle with water which is greater than 60°.

24. A stent according to claim 23 in which the said polymer having a hydrophobic nature is selected from polymethyl methacrylate, polybutyl methacrylate, polyisobutylmethacrylate, olefinic polymers, polybutadiene, polyisoprene, poly(acrylonitrile-butadiene-styrene) or polyvinyl acetate.

25. A stent according to claim 23 in which the said polymer of a hydrophobic nature is polystyrene.

26. A stent according to claim 21, in which the said active ingredient associated with the said polymer coating of a hydrophobic nature is selected from active ingredients having an anti-inflammatory, antiproliferative or antimigratory action and/or immunosuppressants.

27. A stent according to claim 21, in which the said active ingredient associated with the said polymer coating of a hydrophobic nature is 4-[(4-methyl-1-piperazinyl)methyl]-N-[4-methyl-3-[[4-(3-pyridinyl)-2-pyrimidinyl]amino]-phenyl]benzamide methane sulphonate.

28. A stent according to claim 21, in which the quantity of the said active ingredient associated with the said polymer coating of a hydrophobic nature is between 0.0001 mg and 10 mg.

29. A stent according to claim 21, in which the thickness of the said polymer coating of a hydrophobic nature on the stent varies from 0.5 microns to 40 microns, preferably between 1 and 30 microns, even more preferably between 5 and 10 microns.

30. A stent according to claim 21, in which the said active ingredient is released from the said polymer coating of a hydrophobic nature in a time of one month.

31. A stent according to claim 21, in which the active ingredient and the quantity of active ingredient associated with the said two polymer coatings respectively is the same or different.

32. A stent according to claim 21, which further includes a layer of hyaluronic acid covalently bound to the said polymer coating of a hydrophobic nature.

33. A process for obtaining a coated stent comprising the stages of:
a) dissolving the hyaluronic acid ester and the active ingredient in the same organic solvent to obtain a solution,
b) immersing and then removing the stent in the said solution,
c) removing the solvent by evaporation.

34. A process according to claim 33 in which the said organic solvent is a dipolar aprotic solvent.

35. A process according to claim 34, in which the said organic solvent is selected from dimethyl sulphoxide, N-methylpyrrolidone, dimethylformamide or hexafluoroisopropanol.

36. A process according to claim 33, comprising a stage of pre-treatment of the surface of the stent to which a layer of covalently bound hyaluronic acid is applied.

37. A process according to claim 33 in which the said stages a), b), c) are preceded by the following stases in order:
a¹) dissolving the polymer of a hydrophobic nature and the active ingredient in the same organic solvent to obtain a solution or a suspension,
b¹) immersing and then removing the stent in the said solution or suspension,
c¹) removing the solvent by evaporation.

38. A process according to claim 37 in which the said organic solvent is a low-boiling-point solvent having a boiling point at ambient pressure which is below 100° C., preferably below 80° C.

39. A process according to claim 38 in which the said organic solvent is selected from dichloromethane, methylene chloride, acetone, aliphatic hydrocarbons or cyclohexane.

40. A process according to claim 37 comprising a further stage d¹) in which a layer of covalently bound hyaluronic acid is applied to the polymer coating of a hydrophobic nature.

41. Use of a hyaluronic acid ester for the preparation of a coating for a stent for use in angioplasty.

42. Use according to claim 41 in association with a pharmacologically active ingredient.
